(19) United States
(12) Patent Application Publication
(10) Pub. No.: US 2026/0041858 A1
(43) Pub. Date: Feb. 12, 2026

(54) AIRWAY TOPICALIZATION APPLICATOR APPARATUS

(71) Applicant: Julian William deBacker, Etobicoke (CA)

(72) Inventor: Julian William deBacker, Etobicoke (CA)

(21) Appl. No.: 19/283,718

(22) Filed: Jul. 29, 2025

Related U.S. Application Data
(60) Provisional application No. 63/680,718, filed on Aug. 8, 2024.

(51) Int. Cl. A61M 11/02

(52) U.S. Cl. CPC A61M 11/02; A61M 2202/0007; A61M 2202/0241; A61M 2210/1028; A61M 2210/1032

(57) ABSTRACT

An applicator apparatus has a delivery tube with differently curved tube sections along the length of the delivery tube so as to be tailored for airway topicalization during video laryngoscopy. The apparatus aims to enhance the precision and effectiveness of airway topicalization, particularly in scenarios where conventional applicators encounter challenges due to the acute angle of the laryngoscope blade. By conforming to the shape of the video laryngoscope, a sigmoidal shape of the delivery tube facilitates precise targeting of glottic structures and the posterior pharynx. This innovation is particularly beneficial for short procedures requiring endotracheal intubation, as proper topicalization is known to reduce coughing and bucking on extubation, mitigating potential surgical complications and minimizing the aerosolization of respiratory particles and viruses.

This application claims the benefit under 35 U.S.C.119 (e) of U.S. provisional application Ser. No. 63/680,718, filed Aug. 8, 2024.

### FIELD OF THE INVENTION

The present invention relates to an applicator apparatus for mounting onto an endotracheal topical anesthetic container and for subsequent insertion into the airway of a patient for delivering the anesthetic topically to the airway during video laryngoscopy.

### BACKGROUND

Airway topicalization is a critical step in various medical procedures, including endotracheal intubation, bronchoscopy, and other respiratory interventions. Video laryngoscopy has emerged as a valuable tool for airway management, offering improved visualization of the glottic structures and increased likelihood of first-pass success on intubation. However, existing nozzles used for airway topicalization often struggle to effectively target the desired areas due to the acute angle of the laryngoscope blade. This limitation can lead to suboptimal topicalization, increased patient discomfort following extubation, and potentially higher risks of complications related to coughing and bucking on emergence including the aerosolization of respiratory pathogens and particles.

### SUMMARY OF THE INVENTION

According to one aspect of the invention there is provided an applicator apparatus for use with an endotracheal topical anesthesia delivery device having an outlet member through which a metered dose of topical anesthetic is delivered when activated, the applicator apparatus comprising:
a connector arranged for attachment to the outlet member;
a delivery tube including an internal passage communicating therethrough between (i) a proximal end of the delivery tube in communication with the connector to receive the metered dose when the connector is coupled to the outlet member and (ii) a distal end of the delivery tube arranged to dispense the metered dose therefrom; and
a nozzle formed on the distal end of the delivery tube and arranged to disperse the metered dose delivered through the distal end of the delivery tube;
wherein the delivery tube, in a relaxed state, includes (i) a first tube section extending from the distal end and following a distal curvature and (ii) a second tube section between the first tube section and the proximal end in which the second tube section is configured differently from the distal curvature of the first tube section.

The shape of the delivery tube including differing first and second tube sections for airway topicalization during video laryngoscopy offers a significant advancement in airway management. Its innovative design addresses limitations of conventional nozzles, providing improved targeting, reduced patient discomfort, and decreased procedural complications, thereby benefiting both patients and healthcare providers. More particularly, the shape of the delivery tube enhances precise targeting of glottic structures and the posterior pharynx, enhancing airway topicalization effectiveness. The accurate delivery of topical anesthetic also minimizes patient discomfort during procedures like intubation and extubation. Enhanced topicalization also reduces coughing and bucking on extubation, lowering the risk of surgical complications and respiratory aerosolization. The apparatus also seamlessly integrates with existing video laryngoscope systems, requiring no additional modifications to technique for intubation.

The gradual curvature of distal end of the delivery tube closely matches the contour of the video laryngoscope blade so that the distal end of the nozzle can deliver non-aerosolized lidocaine to the patient's airway, while the proximal end features a connector compatible with standard airway topicalization devices. The internal passage of the nozzle is designed to deliver topical anesthetic agents or other medications directly to the target areas within the airway. The outer surface of the nozzle may include markings or indicators to assist healthcare providers in positioning and orienting the device during use.

When used with a video laryngoscope having a curved blade, the first tube section of the delivery tube preferably has a radius of curvature which is substantially identical to a radius of curvature of the curved blade.

In some embodiments, the first tube section and the second tube section lie in a common plane.

The delivery tube preferably further includes an offset section adjacent the proximal end of the delivery tube, in which the offset section protrudes transversely outward from a first plane locating the first tube section of the delivery tube therein.

When the connector includes a connector axis arranged for alignment with an outlet axis of the outlet member, the connector axis is preferably arranged to be upright in orientation less than 45 degrees from vertical when the delivery tube is oriented to extend primarily vertically from the distal end to the proximal end of the delivery tube. More particularly, the connector axis may be less than 45 degrees from vertical when a first plane locating the first tube section therein is vertically oriented and a dispensing axis of the nozzle is oriented horizontally. The connector axis may also be sloped between 5 degrees and 40 degrees from a first plane locating the first tube section therein.

Preferably a discharging axis of the nozzle is oriented within 10 degrees of horizontal when (i) a first plane locating the first tube section of the delivery tube therein is vertically oriented and (ii) a second plane locating the connector axis therein is vertical and perpendicular to the first plane.

The discharging axis of the nozzle may be oriented horizontally when (i) a first plane locating the first tube section of the delivery tube therein is vertically oriented, and (ii) a second plane locating the connector axis therein is vertical and perpendicular to the first plane.

Preferably the nozzle comprises a restricted orifice arranged to dispense the metered dose therethrough as a dispersed mist.

According to a first embodiment, the first tube section and the second tube section may collectively define a sigmoidal shape of the delivery tube between the proximal end and the distal end. Preferably the first tube section and the second tube section defining the sigmoidal shape lie in a common plane in this instance. The first tube section is preferably curved through an arc of at least 90 degrees.

According to a second embodiment, the delivery tube may further include (i) a first intermediate tube section extending from the first tube section and having a first intermediate curvature different from the distal curvature, and (ii) a second intermediate tube section extending between the first intermediate tube section and the second tube section and having a second intermediate curvature different from the distal curvature. The first and second intermediate curvatures may collectively define a sigmoidal shape. Preferably the first and second intermediate curvatures lie in a common plane with the distal curvature of the first tube section. The first intermediate curvature may be in a reverse direction relative to the distal curvature of the first tube section, and the second intermediate curvature of the second intermediate tube section may be in a reverse direction relative to a second curvature of the second tube section.

According to a second aspect of the present invention there is provided a method of delivering a metered dose of topical anesthetic using the applicator apparatus, the method comprising:
connecting the endotracheal topical anesthesia delivery device to the connector at the proximal end of the delivery tube;
inserting the distal end of the delivery tube into an airway of a patient lying in a supine position such that (i) the first tube section and second tube section lie in the sagittal plane. (ii) the nozzle is directed towards a larynx of the patient, and (iii) the delivery device extends generally laterally of a face of the patient, while in said relaxed state of the delivery tube; and
actuating the endotracheal topical anesthesia delivery device to dispense said metered dose.

### BRIEF DESCRIPTION OF THE DRAWINGS

Some embodiments of the invention will now be described in conjunction with the accompanying drawings in which:

FIG. 1 is a perspective view of the anesthetic applicator apparatus according to a first embodiment of the present invention;

FIG. 2 is another perspective view of the applicator apparatus according to the first embodiment of FIG. 1 as the applicator apparatus is mounted onto an anaesthetic delivery device;

FIG. 3 is a schematic view of the applicator apparatus according to the first embodiment of FIG. 1 in use with a video laryngoscope during a laryngoscopy procedure in a neutral orientation of the apparatus aligned with a sagittal plane of a patient;

FIG. 4 is a top view along the line 4-4 in FIG. 3;

FIG. 5 is an end view along the line 5-5 in FIG. 3;

FIG. 6 is a perspective view of the applicator apparatus according to the first embodiment of FIG. 1 shown in first and second deflected positions angularly offset in opposing directions from the neutral position of FIG. 3;

FIG. 7 is a perspective view of the anesthetic applicator apparatus according to a second embodiment of the present invention;

FIG. 8 is top view of the applicator apparatus according to the second embodiment of FIG. 7 in a working orientation for use in a laryngoscopy procedure relative to a supine patient;

FIG. 9 is a rear end view of the applicator apparatus according to the second embodiment of FIG. 7 in the working orientation; and

FIG. 10 is a side elevational view of the applicator apparatus according to the second embodiment of FIG. 7 shown in the working orientation.

In the drawings like characters of reference indicate corresponding parts in the different figures.

### DETAILED DESCRIPTION

Referring to the accompanying figures, there is illustrated an endotracheal topical anesthetic applicator apparatus generally indicated by reference numeral 10. The applicator apparatus 10 is particularly suited for use with an endotracheal anaesthetic delivery device 12.

In the illustrated embodiments, the anaesthetic delivery device 12 comprises a container with a cylindrical body 14 having an outlet member 16 in the form of a stem or tube protruding axially from the top end of the cylindrical body. The outlet member 16 is connected to an internal valve and pumping chamber controlled by axial movement of the stem relative to the container body. More particularly, when the outlet member 16 is depressed axially in an inward direction into the container, the outlet member actuates the pumping chamber to deliver a measured dose of anaesthetic from the device 12 axially through the tubular body of the stem defining the outlet member 16.

The applicator apparatus 10 is also suited for use together with a video laryngoscope 18 in a laryngoscopy procedure. The laryngoscope in the illustrated embodiment includes a generally cylindrical grip handle 20 arranged to be gripped in a hand of a user and a curved blade 22 extending transversely outward from one end of the grip handle 20. The blade 22 is curved towards the distal end of the blade such that a concave side of the blade faces inwardly towards the grip handle. A camera (not shown) is mounted at the distal end of the blade for capturing video images to assist in various procedures such as intubation of the airway.

The applicator apparatus 10 is intended to be inserted into the airway alongside the curved blade 22 of the laryngoscope 18 to extend further into the airway than the blade 22, guided by the video images captured by the camera of the laryngoscope for applying the anaesthetic from the delivery device 12 into the airway of a patient. Although various embodiments of the apparatus 10 are shown in the accompanying figures, the features in common with the various embodiments will first be described.

The applicator apparatus 10 generally includes (i) a connector 24 arranged for releasable attachment to the output member 16 of the anaesthetic delivery device 12, (ii) a delivery tube 26 with an internal passage extending longitudinally therethrough from a proximal end in communication with the connector 24 to an opposing distal end of the delivery tube, and (iii) a nozzle 28 mounted at the distal end of the delivery tube, whereby a metered dose of anaesthetic discharged from the delivery device 12 is directed through the connector 24 into the delivery tube 26 for subsequent discharge through the nozzle 28 which serves to disperse the metered dose as a mist or atomized discharge of fine droplets.

The connector 24 is a cylindrical body having a first passage 30 extending axially into the body from a bottom end of the body. The first passage 30 is open at the bottom and is suitably sized to receive the tube defining the outlet member 16 of the delivery device 12 inserted therein so that the connector is frictionally retained by interference fit relative to the outlet member. The first passage 30 defines a connector axis 31 of the cylindrical body of the connector 24. The connector 24 also includes a second passage 32 communicating radially from an inner end in communication with the inner end of the first passage 30 to an opposing outer end at the cylindrical boundary of the body of the connector.

The first passage 30 and the second passage 32 collectively redirect delivery of the metered dose from the delivery device 12 through the connector body and into the delivery tube which extends radially outward from the cylindrical body in alignment with the second passage such that the second passage 32 is in open communication with the internal passage of the delivery tube 26. The connector 24 is suitably sized so as not to interfere with the body of the container as the stem is depressed to activate the pumping chamber which discharges the measured dose from the delivery device 12.

The nozzle 28 is formed at the distal end of the delivery tube where the delivery tube is increased or enlarged in outer diameter relative to the remainder of the delivery tube extending between the connector 24 and the nozzle 28. The nozzle 28 includes a generally rounded and spherical outer boundary surface to ensure ease of insertion into the airway of the patient. The nozzle 28 defines a restricted orifice 34 in communication with the internal passage of the delivery tube in which the cross-sectional area at the restricted orifice 34 is reduced relative to the passage of the delivery tube. The orifice 34 of the nozzle defines a nozzle discharge axis 35 defining the primary direction of the discharge of the metered dose from the apparatus 10. The orifice 34 is arranged to disperse the metered dose passing through the orifice as a mist or atomised flow of fine droplets of the anaesthetic dispersed into the airway.

The delivery tube 26 is an elongate tube formed of a resilient plastic material having a shape memory which maintains a prescribed shape in a relaxed state of the tube. In the relaxed state, the delivery tube includes (i) a first tube section 36 defining a distal curvature of the delivery tube extending from the distal end towards the proximal end, (ii) a second tube section 38 extending from the first tube section towards the proximal end, and (iii) an offset section 40 extending between the second tube section 38 and the distal end.

The first tube section 36 is immediately adjacent the distal end of the delivery tube and has a radius of curvature that is substantially identical to the radius of curvature of the blade of the video laryngoscope 18 so as to be suited for extending alongside the blade 22 when inserted into the airway. The first tube section 36 lies in a common first plane 37 aligned with the sagittal plane of the patient when in use.

The second tube section 38 is configured differently from the distal curvature of the first tube section. More particularly, the second tube section 38 has a different radius of curvature and curves in a different direction relative to the first tube section. In the illustrated embodiments, the second tube section 38 commonly lies in the first plane 37 of the first tube section 36 but forms a reverse curve that curves in the opposing direction as the first tube section.

The offset section 40 extends from the second tube section 38 to extend laterally outwardly from the common first plane 37 of the first tube section 36 such that the connector 24 connected at the proximal end of the offset section 40 is likewise positioned so as to be spaced laterally outwardly from the first plane of the first tube section. In this instance, when the first tube section 36 is aligned with the sagittal plane of the user, the connector 24 mounted at the proximal end of the offset section 40 is spaced laterally to one side of the sagittal plane.

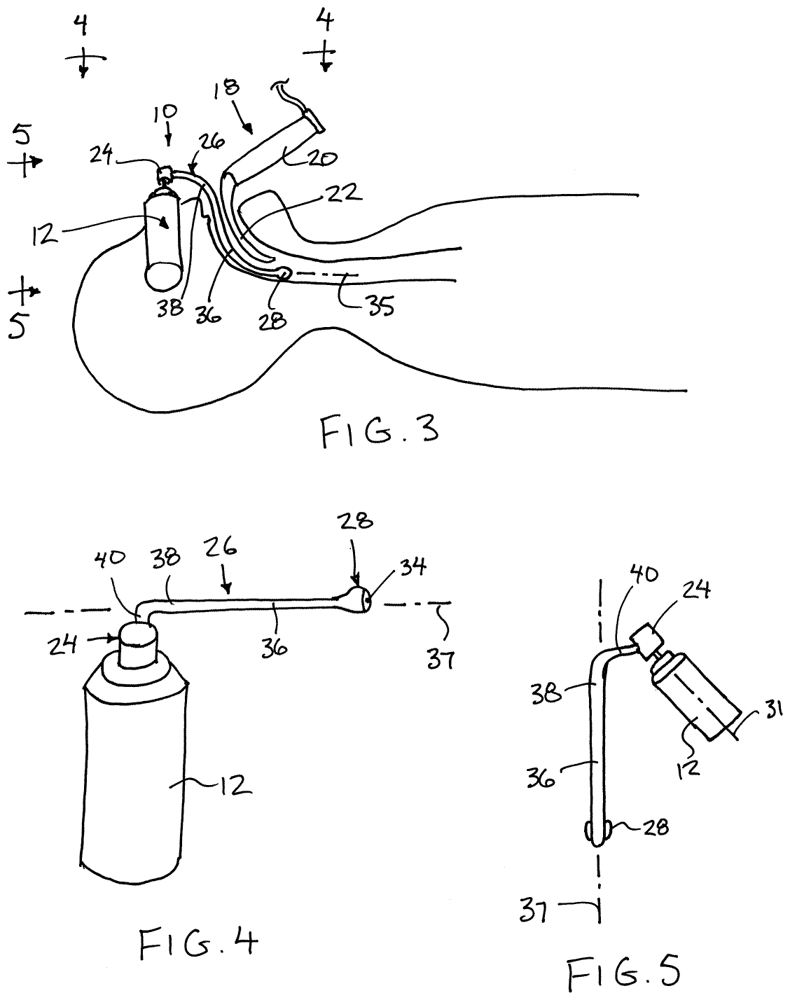

In use, the distal end of the delivery tube is inserted into the airway of the patient such that the first tube section 36 occupies the first plane 37 which is aligned with the sagittal plane. When the first tube section lies in a vertical plane and the delivery tube extends continuously upward from the distal end to the proximal end, the connector at the proximal end is located at the top of the apparatus 10 so as to be spaced above the face of the patient. In a neutral position of the apparatus as shown in FIGS. 3 through 5, in addition to the connector 24 being offset laterally outwardly from the sagittal plane, the connector axis 31 is in an upright orientation while being sloped generally downwardly and laterally outwardly so that the connected delivery device 12 extends similarly downwardly and outwardly along the connector axis from the connector 24.

In the neutral position, the connector axis 31 lies in a second vertical plane that is perpendicular to the first vertical plane locating the first tube section therein. In this neutral position, the connector axis is preferably sloped downwardly and outwardly from the first vertical plane by at least 5 degrees and preferably more than 20 degrees or more than 30 degrees so that the body of the container of the delivery device 12 is sufficiently clear of the face of the patient while remaining sufficiently upright in orientation that the pumping arrangement of the delivery device is functional. To ensure that the container is sufficiently upright, the connector axis is preferably less than 45 degrees and more preferably less than 40 degrees from the second vertical plane.

Furthermore, in the neutral position, the discharging axis of the nozzle 28 is preferably horizontally oriented or oriented at a slope of less than 10 degrees from horizontal. In this manner, the nozzle is well oriented to allow some pivoting movement of the apparatus 10 about a lateral axis oriented perpendicularly to the sagittal plane and the first plane 37 of the first tube section. By rotating the apparatus 10 about the lateral axis through a range of approximately 45 degrees in either direction from the neutral position as shown in FIG. 6, the first tube section 36 remains within the vertically oriented sagittal plane and the connector axis 31 remains approximately within 45 degrees of vertical. Accordingly, the delivery device 12 remains sufficiently upright to be operational through the range of motion while the discharge direction of the nozzle varies from being sloped upwardly and forwardly into the airway (i.e. towards anterior glottis structures) at approximately 45 degrees from horizontal to being sloped downwardly and forwardly into the airway (i.e. towards posterior pharynx) at approximately 45 degrees from horizontal.

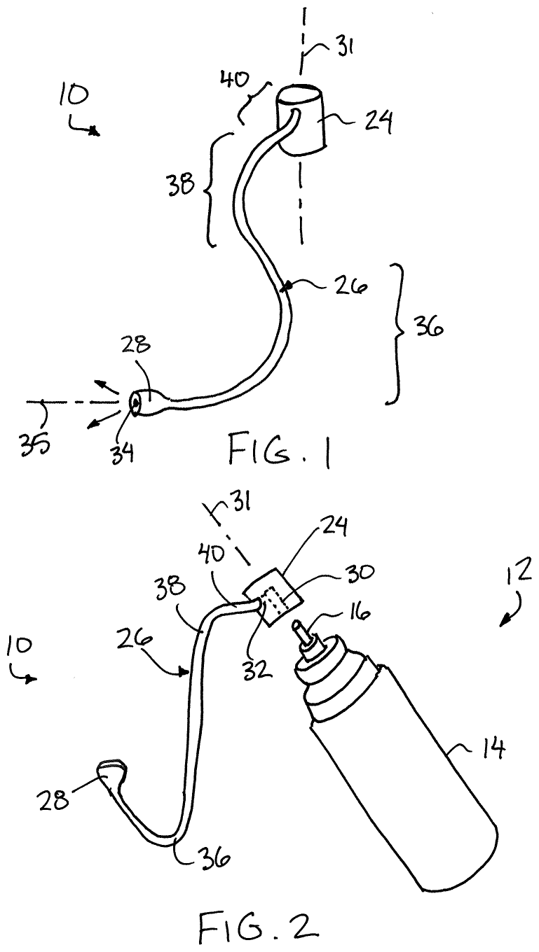

Turning now more particularly to the first embodiment of FIGS. 1 through 6, in this instance the delivery tube consists only of the first tube section 36, the second tube section 38, and the offset section 40 which are continuous with one another between the distal end and the proximal end of the delivery tube. In this instance, the first and second tube sections collectively define a sigmoidal shape of the delivery tube. The first tube section 36 preferably extends through an arc of at least 90 degrees or more so as to occupy the majority of the overall length of the delivery tube. In a variation of the first embodiment, the second tube section 38 may primarily define a reverse curve relative to the first tube section but may deviate laterally outwardly from the first plane 37 containing the first tube section. In this instance, the sigmoidal shape may not necessarily lie entirely within a single plane.

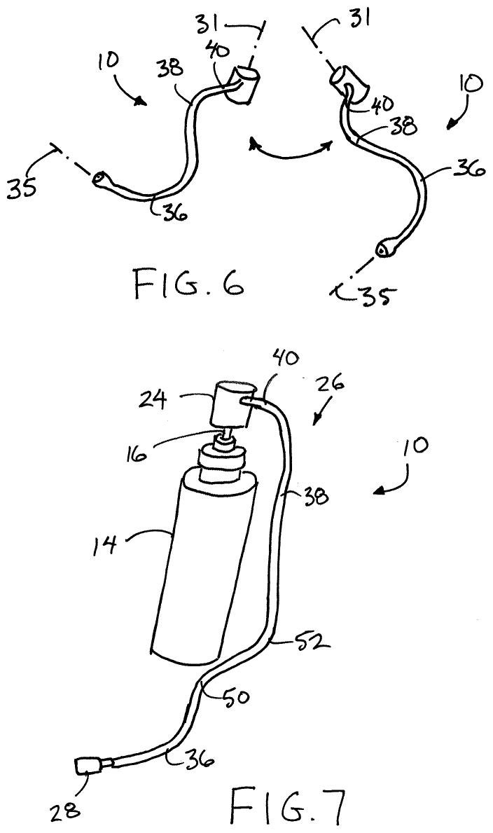

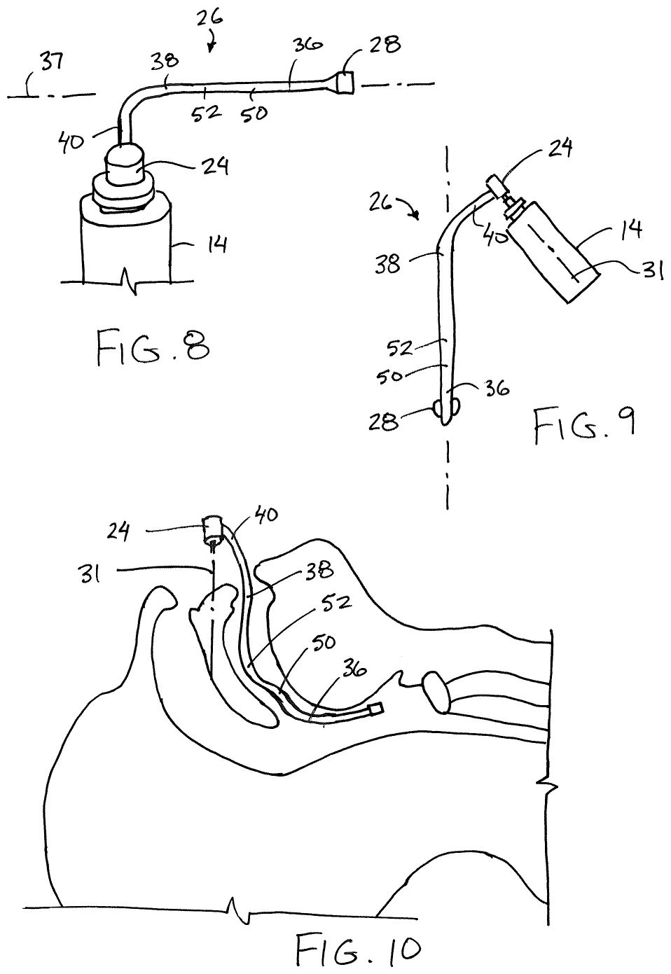

Turning now to the second embodiment of FIGS. 7 through 10, in this instance, the apparatus 10 differs from the previous embodiment in the transition between the first tube section 36 and the second tube section 38. More particularly, in this instance the transition between the first tube section 36 and the second tube section 38 further includes a first intermediate tube section 50 extending from the first tube section and a second intermediate tube section 52 connected between the first intermediate tube section 50 and the second tube section 38.

The first intermediate tube section 50 defines a first intermediate curvature which is in a reverse direction relative to the distal curve of the first tube section 36 while the second intermediate tube section 52 follows a second intermediate curvature which reverses again in direction relative to the first intermediate curvature. In this manner the first intermediate curvature of the first intermediate tube section 50 and the second intermediate curvature of the second intermediate tube section 52 collectively define a sigmoidal shape of a transition between the first tube section 36 and the second tube section 38. The second intermediate curvature of the second intermediate tube section 52 is also in reverse to the second curvature of the adjacent second tube section 38 connected thereto. In this manner, the first tube section 36, followed by the first intermediate tube section 50, then the second intermediate tube section 52, and finally the second tube section 38, collectively follow a sinusoidal-like shape.

The intermediate tube sections 50 and 52 lie in a common plane with the first tube section 36 corresponding to the sagittal plane of the patient when the distal end of the delivery tube is inserted into the airway of the patient in a working orientation as shown in FIG. 10. The second tube section 38 may also lie within the same sagittal plane but is typically straighter than the other curvatures. In some instances, the curvature of the second tube section follows a compound curve including a first curved deflection within the sagittal plane and a second curved deflection within a lateral plane to transition into the generally laterally oriented direction of the offset section 40 connected to the proximal end of the second intermediate tube section.

The various tube sections are sized relative to one another in this instance such that reverse curvature of the first intermediate tube section 50 relative to the first tube section 36 generally aligns with the soft palate of the patient. Accordingly, an intermediate portion of the delivery tube 26 is moved forwardly relative to the previous embodiment to prevent hitting the soft palate and thereby minimize interference of the insertion of the delivery tube into the airway of the patient. In this manner, the nozzle can be better oriented for being directed onto the vocal cords while the delivery tube remains in a relaxed state.

In one exemplary use of the applicator apparatus 10, the apparatus 10 works together with the endotracheal anaesthetic delivery device 12 for anaesthesia of the airway during a laryngoscope procedure, for example intubation of the airway. In this instance, the video laryngoscope is first inserted into the airway as represented schematically in FIG. 3. The applicator apparatus 10 is releasably connected to the output member 16 of the delivery device 12 and the apparatus is primed with anaesthetic from the delivery device by actuating the output member 16 until the internal passages of the apparatus have been filled with anaesthetic. The distal end of the delivery tube of the apparatus 10 is then inserted into the airway such that the first tube section follows closely along the curvature of the blade 22 until the distal end of the apparatus 10 extends past the distal end of the blade 22 as visualized in the images captured by the video laryngoscope. In the resulting working orientation of the apparatus 10, (i) the first tube section and second tube section lie in the sagittal plane. (ii) the nozzle is directed towards a larynx of the patient, and (iii) the delivery device extends generally laterally of a face of the patient, while in said relaxed state of the delivery tube. With the first tube section lying in the vertically oriented sagittal plane, the second tube section and the offset section 40 collectively locate the connector 24 spaced away from the grip handle 20 of the laryngoscope and offset laterally to one side of the sagittal plane such that the container body 14 extending from the connector 24 extends downwardly and laterally outwardly above the face of the user. Sufficient clearance is provided between the delivery device and the face of the user that the apparatus can be pivoted about the lateral axis from the neutral position of FIGS. 3 through 5 in either direction to respective first or second deflected positions shown in FIG. 6. The delivery device 12 can be actuated in any of the first deflected position, the neutral position, or the second deflected position to provide an effective dispersal of the metered dose of anaesthetic into the airway of the patient.

As described above, the applicator apparatus for airway topicalization during video laryngoscopy, generally comprises (i) a curved body with a gradual sigmoidal curvature, (ii) a distal end that allows for wide dispersion of non-aerosolized local anesthetic, (iii) a proximal end with a connector for attachment to airway topicalization devices, (iv) an angulated proximal end that allows the operator to avoid contact with the patient's face and permits a <180 degree rotation of spray without shutting of the delivery system due to gravity, and (v) an internal passage for delivering topical anesthetic agents or medications, (vi) wherein the curvature of the body matches the contour of a video laryngoscope blade, and (vii) wherein the applicator is compatible with standard non-aerosolized lidocaine metered dose delivery systems.

Since various modifications can be made in the invention as herein above described, and many apparently widely different embodiments of same made, it is intended that all matter contained in the accompanying specification shall be interpreted as illustrative only and not in a limiting sense.

1. An applicator apparatus for use with an endotracheal topical anesthesia delivery device having an outlet member through which a metered dose of topical anesthetic is delivered when activated, the applicator apparatus comprising:
a connector arranged for attachment to the outlet member;
a delivery tube including an internal passage communicating therethrough between (i) a proximal end of the delivery tube in communication with the connector to receive the metered dose when the connector is coupled to the outlet member and (ii) a distal end of the delivery tube arranged to dispense the metered dose therefrom; and
a nozzle formed on the distal end of the delivery tube and arranged to disperse the metered dose delivered through the distal end of the delivery tube;
wherein the delivery tube, in a relaxed state, includes (i) a first tube section extending from the distal end along a distal curvature and (ii) a second tube section between the first tube section and the proximal end in which the second tube section is configured differently from the distal curvature of the first tube section.

2. The apparatus according to claim 1 in combination with a video laryngoscope having a curved blade, wherein the first tube section of the delivery tube has a radius of curvature which is substantially identical to a radius of curvature of the curved blade.

3. The apparatus according to claim 1 wherein the first tube section and the second tube section lie in a common plane.

4. The apparatus according to claim 1 wherein the delivery tube further includes an offset section adjacent the proximal end of the delivery tube, the offset section protruding transversely outward from a first plane locating the first tube section of the delivery tube therein.

5. The apparatus according to claim 1 wherein the connector includes a connector axis arranged for alignment with an outlet axis of the outlet member, and wherein the connector axis is arranged to be upright in orientation less than 45 degrees from vertical when the delivery tube is oriented to extend primarily vertically from the distal end to the proximal end of the delivery tube.

6. The apparatus according to claim 5 wherein the connector axis is less than 45 degrees from vertical when a first plane locating the first tube section therein is vertically oriented and a dispensing axis of the nozzle is oriented horizontally.

7. The apparatus according to claim 5 wherein the connector axis is sloped between 5 degrees and 40 degrees from a first plane locating the first tube section therein.

8. The apparatus according to claim 1 wherein the connector includes a connector axis arranged for alignment with an outlet axis of the outlet member, and wherein a discharging axis of the nozzle is oriented within 10 degrees of horizontal when (i) a first plane locating the first tube section of the delivery tube therein is vertically oriented, and (ii) a second plane locating the connector axis therein is vertical and perpendicular to the first plane.

9. The apparatus according to claim 1 wherein the discharging axis of the nozzle is oriented horizontally when (i) a first plane locating the first tube section of the delivery tube therein is vertically oriented, and (ii) a second plane locating the connector axis therein is vertical and perpendicular to the first plane.

10. The apparatus according to claim 1 wherein the nozzle comprises a restricted orifice arranged to dispense the metered dose therethrough as a dispersed mist.

11. The apparatus according to claim 1 wherein the first tube section and the second tube section collectively define a sigmoidal shape of the delivery tube between the proximal end and the distal end.

12. The apparatus according to claim 11 wherein the first tube section and the second tube section defining the sigmoidal shape lie in a common plane.

13. The apparatus according to claim 11 wherein the first tube section is curved through an arc of at least 90 degrees.

14. The apparatus according to claim 1 further comprising:
a first intermediate tube section extending from the first tube section and having a first intermediate curvature different from the distal curvature; and
a second intermediate tube section extending between the first intermediate tube section and the second tube section and having a second intermediate curvature different from the distal curvature.

15. The apparatus according to claim 14 wherein the first and second intermediate curvatures collectively define a sigmoidal shape.

16. The apparatus according to claim 14 wherein the first and second intermediate curvatures lie in a common plane with the distal curvature of the first tube section.

17. The apparatus according to claim 14 wherein the first intermediate curvature is in a reverse direction relative to the distal curvature of the first tube section.

18. The apparatus according to claim 14 wherein the second tube section defines a second curvature and wherein the second intermediate curvature of the second intermediate tube section is in a reverse direction relative to the second curvature of the second tube section.

19. A method of delivering a metered dose of topical anesthetic using the applicator apparatus according to claim 1, the method comprising:
connecting the endotracheal topical anesthesia delivery device to the connector at the proximal end of the delivery tube;
inserting the distal end of the delivery tube into an airway of a patient lying in a supine position such that (i) the first tube section and second tube section lie in the sagittal plane. (ii) the nozzle is directed towards a larynx of the patient, and (iii) the delivery device extends generally laterally of a face of the patient, while in said relaxed state of the delivery tube; and
actuating the endotracheal topical anesthesia delivery device to dispense said metered dose.
